(19) United States
(12) Patent Application Publication
(10) Pub. No.: US 2020/0093637 A1
(43) Pub. Date: Mar. 26, 2020

(54) DEVICE AND OPERATIONAL METHOD FOR PLASMA TREATMENT OF BIOLOGICAL TISSUE

(71) Applicant: Reinhardt Thyzel, Eckental (DE)

(72) Inventor: Reinhardt Thyzel, Eckental (DE)

(21) Appl. No.: 16/582,898

(22) Filed: Sep. 25, 2019

(30) Foreign Application Priority Data
Sep. 26, 2018 (EP) 18 196 744.9

(51) Int. Cl. A61F 9/007

(52) U.S. Cl. CPC A61F 9/0079; A61F 9/00781

(57) ABSTRACT

A device for plasma treatment of biological tissue can comprise a hollow needlelike tubular section with a distal end for applying a light-induced plasma to the tissue, wherein the tubular section comprises subsequent to the distal end an inner plasma chamber configured for generating therein a light-induced plasma. In one embodiment, the plasma chamber is communicatively coupled with a distal, central axial aperture in the distal end, and adjoins, within the tubular section, a light injection section for injecting light, in particular laser light pulses, into the plasma chamber for plasma generation.

### CROSS-REFERENCE TO RELATED APPLICATIONS

The present invention claims priority from European Patent Application No. 18196744.9, filed Sep. 26, 2018, the entire content of which is incorported herein by reference.

### BACKGROUND OF THE INVENTION

### 1. The Field of the Invention

The present invention is directed to a device for the treatment of biological tissue, in particular for in vivo plasma treatment of biological tissue, such as for example the trabecular meshwork of the eye of for example a human being.

### 2. Background and Relevant Art

The trabecular meshwork of the human eye is a kind of porous meshwork and is part of a drainage system for draining eye liquid (humor aqueous) generated by the ciliary bodies. In the healthy eye, the eye liquid flows from the posterior eye chamber through the pupil and the anterior eye chamber and is drained over the trabecular meshwork and the Schlemm's canal arranged downstream the trabecular meshwork.

Disturbance of the drainage of the eye liquid, for example through blockage or congestion of the trabecular meshwork, may lead to an increased intraocular pressure (ocular hypertension), which in turn may cause Glaucoma. Removing the blockage or congestion of the drainage system, in particular the trabecular meshwork, is one possibility to eliminate ocular hypertension, and the occurrence of Glaucoma which would otherwise be caused by continued blockage or congestion of the trabecular meshwork.

Thus, there is a need for devices suitable for in vivo tissue treatment, and appropriate for eliminating blockage in the drainage system of the eye, in particular in the trabecular meshwork of the eye.

### BRIEF SUMMARY OF THE INVENTION

The object of the present invention is thus to provide a respective device and operational method for tissue treatment, in particular a device that is suitable for treating the drainage system, in particular trabecular meshwork, of the eye, for example in connection with avoiding Glaucoma caused by disturbances in the draining of the eye liquid in the region of the trabecular meshwork of the eye.

This object is solved in accordance with the invention by the annexed independent claims. Embodiments of the invention result from the dependent claims and the description below.

In embodiments, a device for the treatment of biological tissue, in particular for in vivo plasma treatment of biological tissue, of for example a human or animal organism is provided. The biological tissue may be the trabecular meshwork of the human eye. The treatment of the trabecular meshwork may involve blockage removal within the trabecular meshwork for the avoidance of or removal of ocular hypertension. Treating the trabecular meshwork with a plasma generated by the device described herein below leads to increased penetrability and permeability of the trabecular meshwork for eye liquid, in turn leading to improved discharge capabilities for eye liquid, such that ocular hypertension may be removed or avoided. Ocular hypertension is one of the most important risk factors for the development of Glaucoma.

In embodiments, the device may comprise a hollow needle-like tubular section with a distal end for applying a light-induced plasma to the tissue. For example, the distal end may be positioned on or on the tissue to be treated, such that a light-induced plasma discharged from the device may impinge the tissue. The needle-like tubular section may be used as a needle for penetrating, for example, surrounding tissue and the tissue to be treated as far as necessary.

For example, the needle-like section may be inserted into the tissue with the distal end ahead such that the distal end can be placed at the tissue to be treated with plasma discharged from the distal end of the device.

In embodiments, the tubular section may comprise subsequent to (or: adjacent to) the distal end an inner plasma chamber. The inner plasma chamber may be configured for generating therein, or at least for inducing (or: seeding) therein, a light-induced plasma.

According to embodiments, the distal end comprises a distal, central axial aperture (in particular: a central axial hole), wherein the plasma chamber is communicatively coupled with the distal, central axial aperture. Further, the plasma chamber adjoins, within the tubular section, a light injection section for injecting light, in particular light pulses, into the plasma chamber for plasma generation (or: formation), to be discharged at least in part, for example as a plasma cloud, out of the plasma chamber for plasma treatment of the tissue.

The aperture is configured for and adapted to enabling light-induced plasma, and possibly shockwaves accompanying the plasma, to propagate through the aperture to the outside. By providing the plasma chamber in connection with the aperture, the plasma generation, in particular the processes of plasma generation, may be confined to the plasma chamber, such that only discharged plasma may interact with the tissue. Using the plasma and associated effects, such as shockwaves, propagating through the aperture may be advantageous for gentle tissue treatment.

In particular, the light injection section, the plasma chamber and the aperture may be arranged successively along the longitudinal axis of the tubular section such that a light pulse, in particular a laser light pulse, may be injected into the plasma chamber from the light injection section. Once the irradiation threshold for plasma formation within the plasma chamber is exceeded, a plasma is generated, by optical breakdown, e.g. by multiphoton avalanche ionization.

The plasma generated or induced within the plasma chamber, for example by nanosecond laser-pulses, evolves and propagates within the plasma chamber, in particular towards the incoming pulse, for example opposite to the light input direction. Due the fact that the plasma chamber defines a confined volume that is communicatively coupled with the environment via the aperture, the plasma can propagate and spread out of the plasma chamber, i.e. discharge out of the plasma chamber. The portion of the plasma propagating and spreading out of the plasma chamber can, at least in part, be used for tissue treatment. Interactions between the plasma and the tissue result in plasma-mediated distortions, such as ablation and disruption, which, if applied to the trabecular meshwork for example result in improved permeability or penetrability for eye fluid. Propagation of the plasma within and out of the plasma chamber may be accompanied by shockwaves generated in connection with an optical breakdown causing the plasma formation.

The primary purpose of the device resides in the plasma-induced (or: plasma-mediated) treatment of tissue. As noted above, the generation of the plasma, e.g. by optical breakdown, in particular laser-induced breakdown, may be accompanied by shockwave formation which may affect plasma propagation, and, beyond that, contribute as such to the tissue treatment. In other words, the plasma-mediated effects for treating the tissue are the intended primary effects for tissue treatment, and secondary effects for example induced by shockwave formation, may be present and may contribute to the overall impact of the treatment.

By providing the plasma chamber, laser light emitted from the light injection section can be confined at least to a large extent to the plasma chamber. A portion of the laser light may escape from the plasma chamber prior to reaching the irradiance threshold for plasma formation.

As soon as the irradiance threshold is reached, the laser energy is absorbed substantially completely for plasma generation, wherein plasma formation in the plasma chamber prevents the laser light from escaping the chamber.

The portion of the light that is able to escape the chamber prior to and/or after plasma formation, has comparatively low energy and low energy density, with only moderate, low or even negligible impact on the tissue. In particular, the effects of the laser light as such on the tissue may, with regard to tissue treatment, may (substantially) be neglected if compared to plasma-mediated tissue treatment.

The device as described herein, providing a plasma discharged from the plasma chamber for tissue treatment, is particularly suitable for treating the trabecular meshwork of the eye. In particular, it has been shown that the plasma-mediated treatment of the trabecular meshwork by using the device as described herein is effective for removing blockage of the trabecular meshwork and for improving permeability of the trabecular meshwork for eye liquid, for example by generating one or more holes or perforating at lease sections of the trabecular meshwork, to thereby reduce the risk of Glaucoma caused by ocular hypertension.

In embodiments, the aperture, the plasma chamber, and the light injection section are arranged coaxially with each other and, in particular, with the longitudinal axis of the tubular section. In particular, the light injection section may face (or: be oriented towards) the distal, central axial aperture, and the normal of the aperture as well as the optical axis of the light injection section may coincide with the longitudinal central axis of the plasma chamber which in turn may coincide with the longitudinal axis of the tubular section. Such an arrangement in particular may provide expedient plasma formation properties, suitable for discharging plasma out of the plasma chamber for tissue treatment. Further, such a design enables simple and compact construction of the device, in particular the tubular section.

In embodiments, the cross section of the aperture measured perpendicular to the longitudinal axis of the plasma chamber or of the tubular section is in the range from 0.05 mm to 0.5 mm, preferably 0.05 mm to 0.3 mm. Such cross sections in particular have been proven advantageous for generating and discharging a plasma for treating the trabecular meshwork for the purpose of enhancing the permeability and penetrability for eye liquid.

In embodiments, the distal end of the tubular section is dome-shaped. Further, the distal end may have an outer rounded tip, wherein a ratio of the outer diameter of the tubular section and the bending radius of the tip end may be at in the range of about 2:1.

In embodiments, the distal end may comprise an inner, axial face that is oriented towards the plasma chamber. The inner axial face may delimit the plasma chamber in axial direction towards the aperture. Further, the axial face may surround the axial aperture, and may, for example, have an annular ring shape.

In embodiments, the axial face may, in axial direction towards the aperture, be conically tapered. In particular, the axial face may form a transitional zone for diameter reduction between the inner circumferential wall of the plasma chamber and the distal aperture. The tapering of the axial face may support plasma formation and/or plasma discharge through the aperture.

Further, the tapering of the axial face may be selected to obtain a particular (or: a desired) shape of the discharged plasma.

In embodiments, the tapering of the axial face may be such that an opening angle of the conically tapered axial face is in the range from 115 to 120 degrees, preferably 118 degrees. Such opening angles in particular have been proven effective for treating the trabecular meshwork for the purpose of improved penetrability and permeability for eye liquid.

In embodiments, the outer diameter of the tubular section, in particular or at least in the region of the plasma chamber, may be in the range between 0.6 mm and 1.0 mm, preferably 0.8 mm. Further, the inner diameter of the tubular section, in particular or at least in the region of the plasma chamber, may be in the range between 0.5 to 0.7 mm, preferably 0.6 mm. Further, the outer and inner diameter of the tubular section may be constant over the whole length of the tubular section, in particular at least over the whole length of the plasma chamber except of course in the region of the tapered axial face where the inner diameter is reduced to the cross section of the aperture.

According to further embodiments, a ratio between the outer diameter and the inner diameter may be in the range of about 4:3.

Such inner and outer dimensions of the tubular section and/or the plasma chamber or parts thereof have been proven advantageous for treating the trabecular meshwork to improve penetrability and permeability for eye liquid.

In embodiments, the wall thickness of the tubular section at the distal end measured parallel to the longitudinal axis of the tubular section is in the range from 0.18 mm to 0.22 mm, preferably 0.2 mm. By this, the distal end may provide sufficient mechanical stability and may provide sufficient material strength to act as a laser absorption target for absorbing laser light.

In embodiments, the axial length of the plasma chamber measured parallel to the longitudinal central axis of the tubular section is at least as large as the inner diameter of the tubular section.

Further, in embodiments the axial length of the plasma chamber may be, at least by a factor of 1.5, in particular of about 1.66, larger than the inner diameter of the tubular section.

In further embodiments, the axial length of the plasma chamber may be, in absolute terms, in the range from 0.5 mm to 1.2 mm, in particular 0.9 mm to 1.1 mm.

Such plasma chamber dimensions have proven advantageous for generating a plasma discharge from the plasma chamber suitable for treating biological tissue, in particular the trabecular meshwork in connection with enhancing penetrability and permeability for eye liquid.

In embodiments, the light injection section has a numerical aperture (in air) in the range from 0.2 to 0.24, in particular 0.22.

According to further embodiments, the light injection section may include as an axial end face facing the plasma chamber, a light exit face, in particular a laser light exit face, of a light guide, such as for example an optical fiber, and/or of a fiber optic, for example associated with a light guide or optical fiber. The light guide may be guided in and arranged coaxially within the tubular section, the plasma chamber and/or the aperture.

In embodiments, the axial end face, in particular the light exit surface, may, in embodiments, be oriented towards the aperture. The light guide may be disposed within the tubular section such that, in particular in the region of the axial end face, the optical axis of the light guide is coaxial with the tubular section, in particular the longitudinal axis of the tubular section, the plasma chamber and/or the aperture.

Further, the light guide, in particular the light exit face, the light exit face, and/or the fiber optic may have a numerical aperture (in air) in the range from 0.2 to 0.24, in particular 0.22. In embodiments, a numerical aperture (in air) of 0.2 to 0.24 may be implemented in connection with an opening angle for emitted light in the range of about 25 degrees.

The suggested configuration of the light injection section and light guide, in particular their arrangement and the numerical aperture, has been proven advantageous in connection with generating the plasma within the plasma chamber and discharging at least a part of the plasma out of the plasma chamber.

In embodiments, the light guide may be arranged adjacent to the plasma chamber, in particular immediately adjacent to the plasma chamber, such that the light exit surface of the light guide faces the inner axial face. The light guide may be configured and arranged such that at least a portion of the light exiting the light exit surface impinges on the inner axial face, to generate or at least to partially contribute to inducing, within the plasma chamber, a plasma by optical breakdown in which volumetric material associated with the inner axial face may act as a light absorption target.

In embodiments, the device may further comprise a tubular extension, in particular a hollow tubular extension, preferably arranged immediately adjacent to the end of the tubular section averted from the distal end. The tubular extension may be arranged coaxially with the tubular section. The tubular extension may have an outer diameter that is larger than the outer diameter of the tubular section. In embodiments, the inner diameter of the tubular extension may substantially correspond to the outer diameter of the tubular section.

In embodiments, the tubular extension may be configured for being inserted and mounted within a recess provided in a mounting head. The mounting head may be configured for being mounted to a handpiece that is suitable for manual handling of the device.

In embodiments, the device may further comprise the handpiece. In such embodiments, the mounting head may be fastened to one end of the handpiece.

Further, a flexible duct for guiding therein the light guide, for example an optical fiber associated with the light injection section, may be attached to the other end of the handpiece. The flexible duct may be disposed at the end averted from the one end to which the mounting head is fastened.

In embodiments, the light guide, in particular the optical fiber, may be fastened within or at the hollow tubular section, the tubular extension, the handpiece and/or the flexible duct by means of at least one adhesive bond. By this, adequate fixation for reliable and uniform plasma generation and formation may be obtained.

In embodiments, the tubular section, including the distal end, and optionally also the tubular extension may be made from titanium or a titanium alloy. The handpiece and/or the mounting head may be made from a plastic material.

In embodiments, the device may further comprise a light source, such as a laser light source. The light source may be configured for and adapted to supplying, via the light guide, light, e.g. laser light, to the light injection section, in particular for emission into the plasma chamber for plasma formation, by suitable optical coupling with the light guide.

The laser light may be a pulsed laser light. Further, the laser light source may be a Q-switched laser, e.g. a Q-switched Nd:YAG laser. The light source may be configured and adapted to generate laser pulses having a pulse width of 8 ns±3 ns, and/or a pulse energy in the range from 6 mJ to 10 mJ. Such operational parameters have been proven advantageous for generating a plasma suitable for treating the trabecular meshwork as described in further detail further above.

Embodiments of the invention relate to the use of a device according to any embodiment described herein for treatment of Glaucoma by removing blockage of the trabecular meshwork of the human or animal eye by applying a light-induced plasma generated within or induced at least in part within the plasma chamber of the device, and discharged out of the plasma chamber for treatment of the trabecular meshwork. Removing blockage may include generating or creating one or more holes and/or perforating the trabecular meshwork at least in part, e.g. in pre-defined sections.

In this connection, the expression “treatment of Glaucoma” shall be understood as the process of removing or avoiding the adverse effects caused by ocular hypertension in turn caused by impaired penetrability and permeability of the trabecular meshwork for eye liquid. The use of a plasma, generated for example by a device according to any embodiment described herein, for the treatment of the trabecular meshwork has proven as an efficient and comparatively gentle way to improve penetrability and permeability for eye liquid.

Embodiments of the invention may relate to a method, in particular an automated method, e.g. a computer-implemented method, of operating a device according to any embodiment described herein, wherein the method may comprise generating, by a light source, in particular a laser light source, such as a Q-switched laser, at least one, preferably a plurality of, light pulses, in particular laser light pulses, and feeding the at least one light pulse by means of a light guide (in particular: through the light guide) to the light injection section to generate a plasma or at least to induce or seed a light-induced plasma within the plasma chamber such that at least a part of the plasma, in particular in form of a plasma cloud, is discharged out of the plasma chamber for tissue treatment, in particular for tissue treatment, for the treatment of Glaucoma.

In particular, the device may be operated such that a plasma is generated or seeded within the plasma chamber, and such that the generated or seeded plasma propagates and is discharged out of the plasma chamber for tissue treatment.

In embodiments of the method, generating the at least one light pulse may comprise generating a laser pulse, in particular a nanosecond laser pulse.

The laser pulse may be generated to have a pulse width of 8 ns±3 ns, and/or a pulse energy in the range from 6 mJ to 10 mJ.

### BRIEF DESCRIPTION OF THE DRAWINGS

Exemplary embodiments of the invention will now be described in connection with the annexed figures in which:

FIG. 1 shows a plan view of a handheld device for in vivo tissue treatment in accordance with the invention;

FIG. 2 shows plan view of a handpiece of the handheld device in a partly exploded view;

FIG. 3 shows an enlarged view of a tool tip of the handpiece;

FIG. 4 shows a perspective view of a tubular tip of the handpiece of FIG. 3;

FIG. 5 shows a plan view of the tubular tip of FIG. 4;

FIG. 6 shows a cross section along the longitudinal axis L of the tubular tip in FIG. 5;

FIG. 7 shows an enlarged view of the section of left-hand tip end of the tubular tip, marked in FIG. 6; and

FIG. 8 also shows the enlarged view of the tip end of the tubular tip, marked in FIG. 6.

### DETAILED DESCRIPTION OF THE PREFERRED EMBODIMENTS

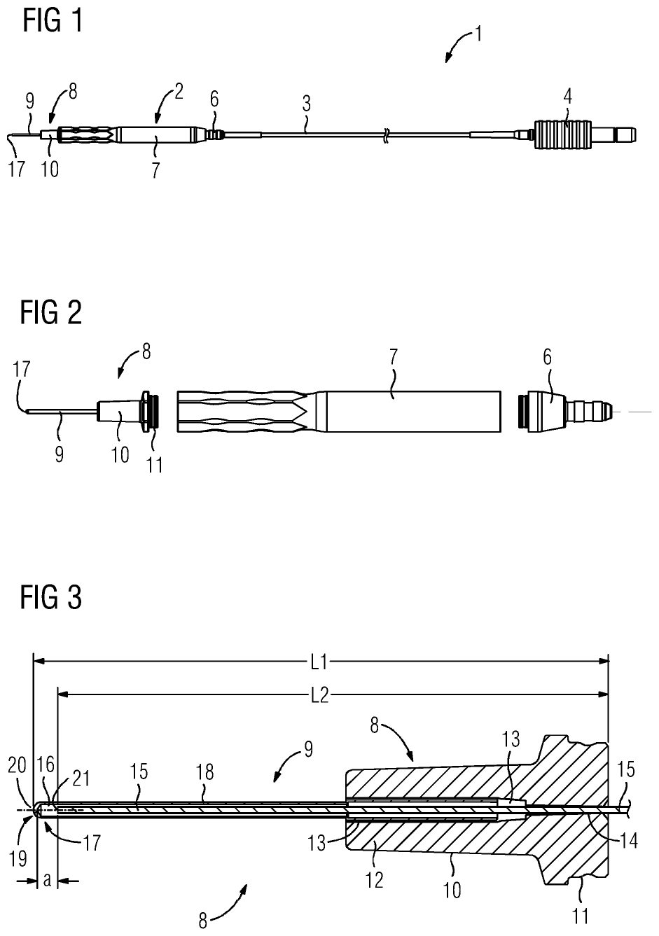

FIG. 1 shows a plan view of a handheld device 1 for use tissue treatment in accordance with the invention, for example for use in in vivo tissue treatment.

The handheld device 1 comprises a handpiece 2 for manual handling. One end of the handpiece 2 is connected with a flexible (in particular: bendable) light supply line 3 which, at the side averted from the handpiece 2, is connected with an connector piece 4 for connecting the handheld device 1 with a light generator or light source (not shown), for example a laser light generator. The laser light generator may comprise a Q-switched Nd:YAG laser adapted to generate laser pulses with a pulse width of 8 ns±3 ns and a pulse energy in the range from 6 mJ to 10 mJ.

As can be seen in greater detail from FIG. 2, a routing element 6 may be provided on a shaft 7 of the handpiece 2, wherein the routing element 6 is configured for avoiding undue bending and nocuous bending radii of the supply line 3 in regions near the end of the shaft 7.

At the side averted from the light supply line 2 and routing element 6, a tool tip 8 is attached (in particular: mounted, for example by adhesive bonding) to the shaft 7 of the handpiece 2. The tool tip 8 comprises, as is also apparent from FIG. 3, a tubular tip 9 comprising a hollow needle-like tubular section, and a mounting head 10.

The mounting head 10 comprises a (male) coupler section 11 (see also FIG. 3) configured for being coupled with one end of the shaft 7 (e.g. a female counterpart), for example by a mechanical bond, and adhesive where appropriate. The mounting head 10 further includes a retainer section 12 with a recess 13 opening at one end of the mounting head 10.

The recess 13 is designed such that the tubular tip 9 can be attached to (in particular: mounted to) the mounting head 10 by inserting one end of the tubular tip 9 into the recess 13. The tubular tip 9 may be secured within the recess 13 by adhesive bonding.

A central bore 14 is provided in the mounting head 10 such that the mounting head includes a central passage for passing through an optical guide, in particular an optical light guide, such as an optical fiber 15, which is shown in FIG. 3.

As will be apparent from the combination of FIG. 1 to FIG. 3, the optical fiber 15 is guided from the tubular tip 9 through the supply line 3 to the connector piece 4. By connecting the connector piece 4 with an appropriate light source (not shown), in particular a laser light source, light, for example in the form of laser light pulses described above, can be guided through the optical fiber from which the pulses may be injected into a chamber 16 provided within the hollow tubular tip 9 at a tip end 17 thereof. Adhesive bonding may be used to attach and fix the optical fiber 15 at components of the device.

The chamber 16 is formed as a hollow space or cavity between the distal end of the optical fiber 15 and an inner distal end section of the tip end 17.

The axial length of the chamber 16 measured parallel to the longitudinal axis L of the tubular tip 9, may for example be in the range from 0.9 mm to 1.2 mm. The axial length of the chamber 16 may for example be defined by the position of the distal end of the optical fiber 15. In particular, during assembly, the optical fiber 15 may be inserted into the tubular tip 9 until a desired distance between the distal end of the optical fiber 15 and the inner distal end section of the tip end 17 is obtained. The distance may be representative of the axial length of the chamber 16.

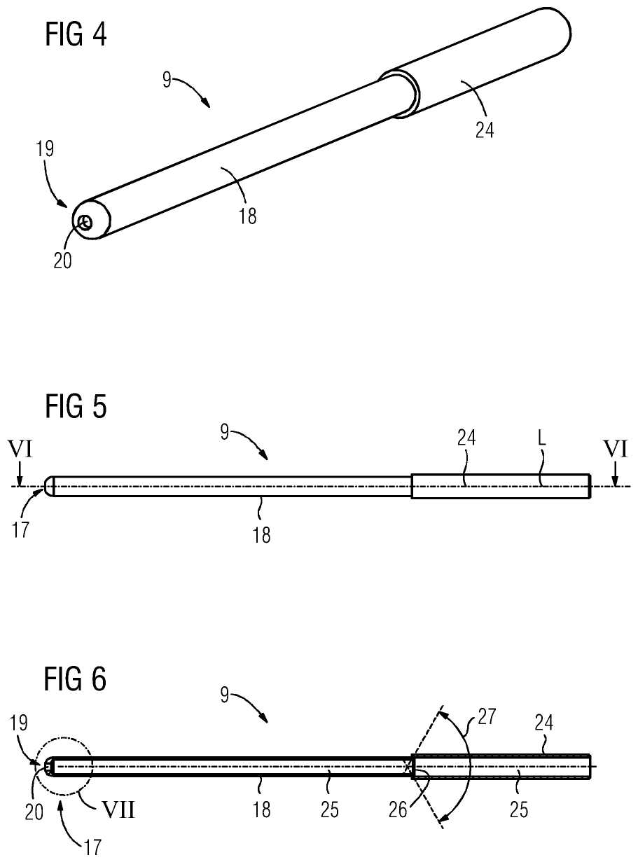

The structure and operation of the hollow tubular tip 9 for the in vivo treatment of biological tissue, such as the trabecular meshwork of the human eye for avoiding Glaucoma, will be described in detail in the following sections, and with reference to FIG. 4 through FIG. 8.

FIG. 4 shows a perspective view of the tubular tip 9. The tubular tip 9 comprises a hollow needle-like (in particular: acicular) tubular section 18 with a distal end 19 suitable for insertion into the tissue to be treated, such as the trabecular meshwork.

The outer diameter of the optical fiber 15 substantially corresponds to the inner diameter of the tubular section 18, wherein the inner diameter of the tubular section may be slightly larger than the diameter of the optical fiber 15 in order to facilitate insertion of the fiber into the tubular tip 9. Such dimensions are advantageous in particular for disposing the optical fiber 15 coaxially with the tubular section 18 and, other parts of the tubular tip 9, such as the chamber 16.

As already mentioned, the optical fiber 15 may be fixed within the tubular section 18, in particular the tubular tip 9, by adhesive bonding, wherein the adhesive bonding may be applied such that a liquid- and/or gas- or air-tight seal between the outer surface or an outer circumference of the optical fiber 15 and the inner surface of one or more corresponding components surrounding the optical fiber 15, such as the tubular section 18, the supply line 3 etc., is obtained, for example at one or more particular locations in lengthwise direction of the optical fiber 15. By this, the optical fiber 15 can be firmly held within the device components, and the channel in which the optical fiber is guided can be protected against environmental influences, wherein undesired substances, such as liquids, can be prevented from infiltrating the channel.

Such adhesive bonding and similar fixing techniques for the optical fiber 15 are possible because the plasma-induced treatment of tissue enabled by the proposed device may be carried out without the need to rinse or drain the tissue to be treated, or to chamber 16 and discharged therefrom, may be carried out without supplying or removing auxiliary or additional liquids or substances through the tubular section 18, in particular the tubular tip 9. Specifically, the device may be designed without additional or auxiliary inner channels for rinsing, draining or extracting liquid(s), debris or other substances from the treatment area, which is advantageous with regard to simplified design and manufacture, and, beyond that, contributes to simplified operation. For the reason that the operation of the device results in a plasma generated (or: induced) in the chamber 16, the chamber 16 is referred to herein also as plasma chamber.

Directly adjacent to the distal end 19, the tubular section 18 includes the inner plasma chamber 16, configured for generating therein a light-induced, in particular laser-light induced, plasma, for example by optical breakdown.

The plasma may be generated for example by emitting a laser pulse (not shown) into the plasma chamber via the optical fiber 15, which may have a numerical aperture of 0.22.

As soon as the energy of the laser pulse, for example with regard to the time-profile of the laser pulse, exceeds an irradiance threshold, non-linear absorption processes may lead to laser-induced breakdown and plasma formation (or: generation). The plasma evolves and propagates within the plasma chamber, and is, based on the design suggested herein, at least in part discharged from the plasma chamber 16, which will be described in greater detail below. The plasma formation may be accompanied by a shockwave that may also be discharged from the plasma chamber 16. Regarding the impact of the shockwave, reference to the discussion further above is made.

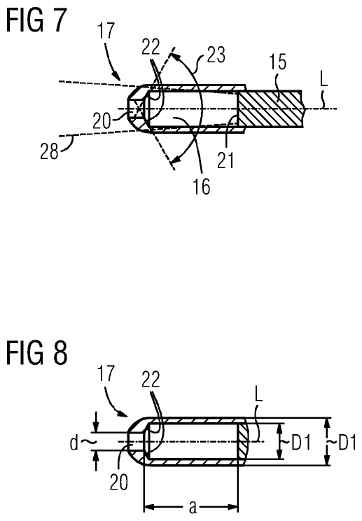

As may be inferred from FIG. 3, FIG. 7, and FIG. 8, the inner plasma chamber 16 is located adjacent to the distal end 19 of the tubular section 18, and is communicatively, in particular fluidly, coupled with a distal, central axial aperture 20 in the distal end 19 of the tubular section 18. In the assembled state, the aperture 20, the tubular section 18, the plasma chamber 16 and the optical fiber 15, as far as guided within the tubular section in the region of and near the plasma chamber 16, are arranged coaxially with one another.

The plasma chamber 16 defines a contiguous inner space or compartment, and is arranged immediately adjacent to a light injection section 21 (see FIG. 3, FIG. 7, FIG. 8) at the distal end of the optical fiber 15. The plasma chamber 16 may be rotationally symmetric with regard to the longitudinal axis L, and may have for example a tubular, in particular cylindrical shape.

The light injection section 21 (in particular: light injection area) in the shown example embodiment is located fully within the tubular section at a pre-defined distance from the central axial aperture 20 or distal inner wall of the distal end 19.

The light injection section 21 in the example embodiment shown in the figures includes as an axial end face facing the plasma chamber 16, a light exit face of the optical fiber 15. The optical fiber 15 is disposed within the tubular section such that the optical axis of the optical fiber coincides with the longitudinal axis L, i.e. is coaxial with the longitudinal axis L of the tubular section 18, in particular the plasma chamber 16.

In particular, the plasma chamber 16, the central axial aperture 20, and the light injection section 21 are configured such that a light-induced plasma generated within the plasma chamber 16 by light injection, in particular laser light injection, into the plasma chamber 16 at least in part is discharged from the plasma chamber 16 through the central axial aperture 20.

During ordinary operation, the part of the plasma discharged from the plasma chamber 16, impinges on the tissue located near or in front of the aperture 20, and the tissue may be modified (or: treated) by plasma absorption. Plasma absorption may be accompanied by shockwave absorption resulting from shockwaves generated in connection with plasma formation. Applied to the trabecular meshwork of the eye, the tissue modification by plasma absorption as suggested herein in particular is such that penetrability and permeability of the trabecular meshwork may be improved such that ocular hypertension as far as caused by blockage of the trabecular meshwork may be eliminated.

The axial length a of the plasma chamber 16 (see FIG. 8) measured along the central longitudinal axis L of the tubular section 18 is at least as large as the inner diameter of the tubular section 18. For example, the axial length a of the plasma chamber 16 may be larger than the inner diameter D1 of the tubular section 18 at least by a factor of 1.5, for example by a factor of about 1.66.

According to exemplary embodiments, the axial length of the plasma chamber may be 1 mm±0.2 mm, in cases where the inner diameter D1 is in the range from 0.3 mm to 0.6 mm. Further, the given dimensions may be used in connection with a laser source that is adapted to generate laser pulses having a pulse length of 8 ns±3 ns and a pulse energy of 6 mJ to 10 mJ for plasma generation. In such exemplary embodiments, plasma generated within the plasma chamber and discharged therefrom may be used for treating the trabecular meshwork as described herein.

As may be inferred from FIG. 4 through FIG. 8, the distal end 19 of the tubular section 18 may be dome-shaped, and may have an outer rounded tip. Such a structure is advantageous with regard to discharging a plasma cloud from the plasma generated within the plasma chamber 16 outwards, and at the same time provides satisfactory properties with regard to tissue insertion penetration, and interaction.

The ratio of the outer diameter D2 of the tubular section and the bending radius of the tip end 17 may for example be in the range of about 2:1.

Regarding absolute geometrical dimensions, in exemplary embodiments, the outer diameter D2 of the tubular section may be in the range of about 0.8 mm. Further, the inner radius D1 may be in the range of about 0.6 mm. Yet further, the bending radius of the tip end 17 or the distal end 19 may be in the range of about 0.4 mm.

The central axial aperture 20 may be configured such that the inner diameter D1 of the tubular section 18 is 2 to 12 fold larger than the cross section of the central axial aperture 20 measured perpendicular to the longitudinal axis L.

In exemplary embodiments, and expressed in absolute terms, the diameter of the central axial aperture 20 may lie in the range from 0.05 mm to 0.5 mm, for example 0.3 mm.

The geometrical dimensions as described beforehand have proven advantageous for discharging the plasma, in particular an appropriate portion of the plasma, out of the plasma chamber 16 such that appropriate treatment of tissue, for example the trabecular meshwork, may be obtained.

As described above, the discharge of a plasma cloud through the central axial aperture 20 and/or the plasma formation within the plasma chamber, may be accompanied by an acoustic shockwave. The shockwave may pass through the central axial aperture 20, in particular subsequent to discharging plasma out of the plasma chamber, wherein the shockwave may contribute to tissue modification as described above.

The interaction of the plasma and the tissue, such as the particular trabecular meshwork, and the interaction of the acoustic wave with the trabecular meshwork increases, for example in connection with using laser pulses as defined further above, the porosity of the trabecular meshwork, i.e. the penetrability and permeability of the trabecular meshwork for eye liquid. By this, ocular hypertension generated by blocked and congested areas of the trabecular meshwork may be eliminated, thereby reducing the risk for Glaucoma.

As may be inferred from FIG. 7 and FIG. 8, the distal end 19 comprises an inner, axial face 22 that is oriented towards the plasma chamber 16. The inner axial face 22 in the embodiment shown in connection with the figures surrounds the inner aperture area of the central axial aperture 20, and has an annular ring shape.

The inner axial face 22 may act as a target for a part of the laser light 28 emitted from the light injection section 21 and the optical fiber 15, wherein the laser light 28 is emitted under a particular opening angle, which is associated with a predetermined numerical aperture, which may be in the range of about 0.22.

Specifically, and as may be inferred from FIG. 3, the optical fiber 15 is arranged adjacent to the plasma chamber 16 such that the light exit surface, i.e. the light injection section 21, faces the inner axial face 22. By this, light emitted from or exiting the light exit surface at least in part may be directed towards and impinge on the inner axial face 22, which may contribute to plasma formation within the plasma chamber 16.

The plasma within the plasma chamber 16 may be generated by laser absorption associated with an optical breakdown, wherein volumetric material associated with the inner axial face 22, and/or of the tubular section in the region of the inner axial face 22, may act at least in part as a light absorption seed for plasma formation.

Regarding the inner axial face 22, the plasma generation may be improved if, as with the example embodiment shown in FIG. 7, the axial face is conically tapered in axial direction towards the aperture. Further, it is advantageous for plasma generation that the opening angle 23 of the conically tapered axial face 22 is in the range from 115 to 120 degrees, for example 118 degrees.

As may be inferred from FIG. 4 to FIG. 6, a hollow tubular extension 24 may be provided in addition to the tubular section 18. The hollow tubular extension 24 is arranged immediately adjacent to the end of the tubular section 18, averted from the distal end 19. Further, the tubular extension 24 is arranged coaxially with the tubular section 18, to form, in combination with the tubular section 18, a coherent (or: continuous) inner channel 25 ranging from the distal end 19 to the end of the tubular extension 24 averted from the distal end 19. The inner channel 25 is configured for accommodating and guiding therein the optical fiber 15, as shown for example in FIG. 3.

As may be inferred from FIG. 3, the tubular extension 24 is provided and configured for being inserted and mounted in the recess 13 of the mounting head 10. An inner shoulder 26 may be provided at the inner wall in the transitional area between the tubular section 18 and the tubular extension 24. The inner shoulder 26 may define a conical tapering of the inner diameter, wherein a further opening angle 27 of the conically tapered inner surfaces of the inner shoulder 26 may be in the range of 115-120 degrees, in particular 118 degrees. Such a shoulder 26 may be beneficial with regard to inserting the optical fiber 15 in the course of assembling the handheld device 1.

The tubular section 18, including the distal end 19, and optionally also the tubular extension 24, are preferably made from titanium or a titanium alloy.

Some further aspects that have been proven advantageous for generating the plasma, and optionally the pressure waves or shockwaves in connection with plasma formation, in particular suitable for treating Glaucoma as described further above, are given below.

According to exemplary embodiments, it has been proven advantageous that the outer diameter D2 of the tubular section 18, in particular of the plasma chamber 16, is in the range between 0.6 mm and 1.0 mm, for example 0.8 mm. Further, the inner diameter D1 of the tubular section 18, in particular of the plasma chamber 16, may advantageously lie in the range from 0.5 to 0.7 mm, for example 0.6 mm. A ratio between the outer diameter and the inner diameter measured in radial direction relative to the longitudinal axis L may for example be 4:3.

In embodiments, it has been proven advantageous that the average material thickness t measured in axial direction, i.e. parallel to the longitudinal axis L, at the distal end 19 is at least twice the material thickness of the tubular section 18 and/or plasma chamber 16 measured in radial direction thereof. The material thickness t may for example be about 0.2 mm.

The above discussion reveals that the suggested device that enables plasma formation in an inner plasma chamber, and discharge of at least a part of the plasma out of the plasma chamber for tissue treating is suitable for solving the underlying problem.

### REFERENCE SIGNS

1 handheld device
2 handpiece
3 supply line
4 connector piece
5 tool tip
6 routing element
7 shaft
8 tool tip
9 tubular tip
10 mounting head
11 coupler section
12 retainer section
13 recess
14 bore
15 optical fiber
16 plasma chamber
17 tip end
18 tubular section
19 distal end
20 central axial aperture
21 light injection section
22 inner axial face
23 opening angle
24 tubular extension
25 inner channel
26 inner shoulder
27 further opening angle
28 laser light
a axial length of the plasma chamber
d cross section of the aperture
D1 inner diameter of the tubular section
D2 outer diameter of the tubular section
L1 axial length
L2 length
L longitudinal axis
t wall thickness at the tip end

I claim:

1. A device for the treatment of biological tissue, in particular for plasma treatment of biological tissue, of for example a human organism, such as for example the trabecular meshwork of the human eye, the device comprising:
a hollow needle-like tubular section with a distal end for applying a light-induced plasma to the tissue;
wherein:
the tubular section comprises subsequent to the distal end an inner plasma chamber configured for generating therein a light-induced plasma,
the plasma chamber is communicatively coupled with a distal, central axial aperture in the distal end, and adjoins, within the tubular section, a light injection section for injecting light, in particular laser light pulses, into the plasma chamber for plasma generation.

2. The device according to claim 1, wherein the aperture, the plasma chamber, and the light injection section are arranged coaxially with each other, in particular with the longitudinal axis (L) of the tubular section.

3. The device according to claim 1, wherein the cross section of the aperture measured perpendicular to the longitudinal axis (L) of the plasma chamber or of the tubular section is in the range from 0.05 mm to 0.5 mm, preferably 0.05 mm to 0.3 mm.

4. The device according to claim 1, wherein the distal end of the tubular section is dome-shaped, and optionally has an outer rounded tip.

5. The device according to claim 1, wherein the distal end comprises an inner, axial face that is oriented towards the plasma chamber, wherein the inner axial face optionally surrounds the axial aperture, and further optionally has an annular ring shape.

6. The device according to claim 5, wherein:
the inner axial face is, in axial direction towards the aperture, conically tapered; and
an opening angle of the conically tapered axial face optionally is in the range from 115 to 120 degrees, preferably 118 degrees.

7. The device according to claim 1, wherein:
the outer diameter (D2) of the tubular section, in particular or at least in the region of the plasma chamber, is in the range between 0.6 mm and 1.0 mm, preferably 0.8 mm;
the inner diameter (D1) of the tubular section, in particular or at least in the region of the plasma chamber, is in the range between 0.5 to 0.7 mm, preferably 0.6 mm; and
a ratio between the outer diameter (D2) and the inner diameter (D1) preferably is 4:3.

8. The device according to claim 1, wherein the wall thickness (t) of the tubular section at the distal end measured parallel to the longitudinal axis (L) of the tubular section is in the range from 0.18 mm to 0.22 mm, preferably 0.2 mm.

9. The device according to claim 1, wherein:
the axial length (a) of the plasma chamber measured parallel to the longitudinal central axis (L) of the tubular section is at least as large as the inner diameter (D1) of the tubular section, preferably at least by a factor of 1.5, in particular a factor of about 1.66, larger than the inner diameter (D1) of the tubular section (18); and/or
the axial length (a) of the plasma chamber is in the range from 0.5 mm to 1.2 mm, in particular 0.9 mm to 1.1 mm.

10. The device according to claim 1, wherein the light injection section has a numerical aperture in the range from 0.2 to 0.24, in particular 0.22.

11. The device according to claim 1, wherein:
the light injection section includes as an axial end face facing the plasma chamber, a light exit face, in particular a laser light exit face, of a light guide, in particular an optical fiber, and/or of a fiber optic associated with a light guide;
the light guide is guided in and arranged coaxially within the tubular section, the plasma chamber and/or the aperture;
optionally, the axial end face, in particular the light exit face, is oriented towards the aperture;
the light guide is disposed within the tubular section such that the optical axis of the light guide is coaxial with the tubular section, the plasma chamber and/or the aperture; and
further optionally, the light guide, the light exit face, and/or the fiber optic has a numerical aperture in the range from 0.2 to 0.24, in particular 0.22.

12. The device according to claim 5, wherein:
the light guide is arranged adjacent to the plasma chamber such that the light exit surface faces the inner axial face, and, preferably, such that at least a portion of the light exiting the light exit surface impinges on the inner axial face, to generate or at least partially contribute to inducing, within the plasma chamber, a plasma by optical breakdown in which volumetric material associated with the inner axial face acts as a light absorption target.

13. The device according to claim 1, further comprising:
a tubular extension arranged immediately adjacent to the end of the tubular section averted from the distal end;
wherein:
the tubular extension is arranged coaxially with the tubular section;
the tubular extension is configured for being inserted and mounted in a recess of a mounting head for mounting to a handpiece for manual handling of the device, wherein the device optionally further comprises the handpiece, wherein the mounting head is optionally fastened to one end of the handpiece; and
a flexible duct for guiding therein a light guide is optionally attached to the other end of the handpiece.

14. The device according to claim 1, wherein the light guide, in particular the optical fiber, is fastened within or at the hollow tubular section, the tubular extension, the handpiece and/or the flexible duct by means of at least one adhesive bond.

15. The device according to claim 1, wherein the tubular section, including the distal end, and optionally also the tubular extension, are made from titanium or a titanium alloy.

16. The device according to claim 1, further comprising:
a light source, in particular laser light source, configured for supplying light, in particular laser light, to the light injection section;
the laser light source is optionally a Q-switched laser, in particular a Q-switched

17. A method of using a device for the treatment of biological tissue, in particular for plasma treatment of Glaucoma using a device comprising:
a hollow needle-like tubular section with a distal end for applying a light-induced plasma to the tissue;
wherein:
the tubular section comprises subsequent to the distal end an inner plasma chamber configured for generating therein a light-induced plasma,
the plasma chamber is communicatively coupled with a distal, central axial aperture in the distal end, and adjoins, within the tubular section, a light injection section for injecting light, in particular laser light pulses, into the plasma chamber for plasma generation,
wherein the method comprises:
removing blockage of the trabecular meshwork of the human or animal eye by applying a light-induced plasma generated within or induced at least in part within the plasma chamber, and discharged out of the plasma chamber for treatment of the trabecular meshwork.

18. A method of operating a device for the treatment of biological tissue, in particular for plasma treatment of biological tissue, of for example a human organism, such as for example the trabecular meshwork of the human eye, the device comprising:
a hollow needle-like tubular section with a distal end for applying a light-induced plasma to the tissue;
wherein:
the tubular section comprises subsequent to the distal end an inner plasma chamber configured for generating therein a light-induced plasma,
the plasma chamber is communicatively coupled with a distal, central axial aperture in the distal end, and adjoins, within the tubular section, a light injection section for injecting light, in particular laser light pulses, into the plasma chamber for plasma generation,
wherein the method comprises:
generating, by a light source, at least one, preferably a plurality of, light pulses, in particular laser light pulses; and
feeding the at least one light pulse by means of the light guide to the light injection section to generate a plasma or at least to induce a light-induced plasma within the plasma chamber, such that at least a part of the plasma, in particular in form of a plasma cloud, is discharged out of the plasma chamber for tissue treatment.

19. The method of claim 18, wherein generating the at least one light pulse comprises generating a laser pulse, in particular a nanosecond laser pulse, preferably with a pulse width of 8 ns±3 ns, and/or with a pulse energy in the range from 6 mJ to 10 mJ.
